(19) United States
(12) Patent Application Publication
(10) Pub. No.: US 2018/0333544 A1
(43) Pub. Date: Nov. 22, 2018

(54) ADAPTER FOR A NEEDLE ASSEMBLY

(71) Applicant: SANOFI, Paris (FR)

(72) Inventor: Markus Ploch, Frankfurt am Main (DE)

(21) Appl. No.: 15/546,224

(22) PCT Filed: Jan. 29, 2016
(86) PCT No.: PCT/EP2016/051931

§ 371 Date: Jul. 25, 2017

(30) Foreign Application Priority Data
Jan. 29, 2015 (EP) 15305106.5

(51) Int. Cl. A61M 5/34; A61M 5/24; A61M 5/32

(52) U.S. Cl. CPC A61M 5/345; A61M 5/2466; A61M 5/3293; A61M 2207/00; A61M 5/344; A61M 5/348; A61M 5/347

(57) ABSTRACT

The disclosure relates to an adapter for attaching a needle assembly to a cartridge holder. The adapter comprises at least one longitudinal arm adapted to engage a respective longitudinal slot in the cartridge holder, such that the adapter is slidable relative to the cartridge holder. The at least one arm has an external thread segment adapted to engage an internal thread of a needle hub of a needle assembly. The at least one arm is adapted to allow for rotationless disengaging the thread segment from the internal thread.

### CROSS REFERENCE TO RELATED APPLICATIONS

The present application is the national stage entry of International Patent Application No. PCT/EP2016/051931, filed on Jan. 29, 2016, and claims priority to Application No. EP 15303106.5, filed in on Jan. 29, 2015, the disclosures of which are expressly incorporated herein in entirety by reference thereto.

### TECHNICAL FIELD

The disclosure relates to an adapter for a needle assembly.

### BACKGROUND

In injection devices, there is a trend toward the use of smaller and smaller gauge needles typically used for self-injection. While a small gauge needle typically reduces the pain associated with the procedure, their use can result in an increased risk of partial or complete occlusion of the needle, especially when used in conjunction with higher concentrated insulins.

One potential cause of needle occlusion is solidification of the drug formulation within the inner bore of the needle. This may occur if the needle is left in-situ following use, or if a user fits a needle in preparation for taking an injection later that day. It is known that particular drug formulations, especially those that are water-based, may be more at risk of solidification in particular storage situations. One such example might be higher concentration insulin formulations. It may be therefore understood as a requirement with crucial impact to the overall reliability of self-injection to attach an unused needle immediately before injection.

There remains a need to help users to easily comply with the aforementioned requirement.

### SUMMARY

An adapter is provided for attaching a needle assembly to a cartridge holder, the adapter comprising at least one longitudinal arm adapted to engage a respective longitudinal slot in the cartridge holder, such that the adapter is slidable relative to the cartridge holder, the at least one arm having an external thread segment adapted to engage an internal thread of a needle hub of a needle assembly, wherein the at least one arm is adapted to allow for rotationless disengaging the thread segment from the internal thread. Avoiding rotation facilitates detachment and, if applicable attachment, of the needle assembly, in particular for users with reduced dexterity.

In an exemplary embodiment, at least two arms are provided, at least two of the arms interconnected to form one or more pairs of connected arms. The external thread is thus distributed over more arms providing for a more secure attachment of the needle assembly on the adapter.

In an exemplary embodiment, at least two arms or two pairs are interconnected by one or more spring arms at a distal end, the spring arms pull the distal end of the arms or pairs together thus reducing a diameter of a screw thread defined by the thread segments to become smaller than a nominal diameter corresponding to the internal thread of the needle hub of the needle assembly. The needle assembly can thus be easily attached to or detached from the adapter without rotation. If after attachment of the needle assembly, the arms or pairs are moved apart against the bias of the spring arm, the needle assembly is secured on the adapter and cannot be pulled off unless the spring arm is allowed to relax.

In an exemplary embodiment, the thread segment exhibits a saw tooth profile comprising a proximal ramped side and a distal transversal side. This may allow screwing the needle assembly onto the adapter and permit it to be pulled off without rotation.

In an exemplary embodiment, the adapter is part of a drug delivery device, further comprising a cartridge holder adapted to receive a drug cartridge, the cartridge holder comprising an interface arranged to receive the adapter, the interface comprising a number of longitudinal slots corresponding to the number of arms of the adapter, the slots adapted to guide the arms such that the adapter is slidable relative to the cartridge holder in a distal direction and in a proximal direction and splined to the cartridge holder. This may allow the adapter to assume different positions relative to the cartridge holder which may be used to allow, cause or prevent engagement or disengagement of the needle assembly to or from the adapter.

In an exemplary embodiment, the at least one arm exhibits a dove tail cross section being wider on a radially inwards pointing side than on a radially outwards pointing side, wherein the at least one slot comprises a dove tail cross section corresponding to the cross section of the at least one arm. The arm is thus prevented from radially outwardly disengaging the slot.

In an exemplary embodiment, the cartridge holder comprises at least one resilient arm with a ramped snap hook and a radially outwardly protruding cam, wherein in a relaxed position of the resilient arm the cam protrudes from an outer surface of a body of the cartridge holder, wherein the snap hook is adapted to engage the adapter for locking it in a snapped-in position. In the snapped-in position, the internal thread of the needle assembly may be secured to the adapter and a proximal tip of a needle of the needle assembly may be positioned to pierce a septum of a cartridge held in the cartridge holder.

In an exemplary embodiment, when the adapter is in an extended position distally from the snapped-in position, the spring arms pull the distal end of the arms or pairs together thus reducing the diameter of the screw thread defined by the thread segments to become smaller than the nominal diameter corresponding to the internal thread of the needle hub of the needle assembly. In this position, the needle assembly may be pushed onto or pulled off the adapter without rotation. In the extended position, the proximal tip of the needle is axially spaced from the septum such that the needle does not have to be precisely centered in the septum when attaching it to the cartridge which is required when the needle assembly is screwed on.

In an exemplary embodiment, a distal face of the cartridge holder is arranged to abut the spring arm opposing movement of the adapter from the extended position in the proximal direction relative to the cartridge holder The distal ends of the arms or pairs are pulled apart against the bias of the spring arms as the arms ride up the slots when the adapter is moved towards the snapped-in position causing the external thread formed by the thread segments to take up its nominal diameter corresponding to the internal thread of the needle hub. The needle hub is thus fully pushed onto the adapter before the threads engage.

In an exemplary embodiment, the distal face is adapted to abut the at least one spring arm straightening it against its bias thereby pushing the thread segments further apart contributing to causing the external thread formed by the thread segments to take up its nominal diameter.

In an exemplary embodiment, the drug delivery device further comprises a cap adapted to be assembled over the cartridge holder thereby engaging the cam deflecting the resilient arm radially inwards thus disengaging the snap hook from the adapter allowing it to return into the extended position due to tension stored in the spring arms thereby reducing the diameter of the screw thread defined by the thread segments to become smaller than the nominal diameter.

The needle assembly is thus automatically removed from the cartridge and released from the adapter upon assembly of the cap.

In an exemplary embodiment, at least one leaf spring is grounded distally in the cartridge holder and proximally in the adapter, wherein in a relaxed state the leaf spring has a curved shape with a first curve radius biasing the adapter in the distal direction, wherein in the relaxed state the leaf spring protrudes radially outwardly beyond a surface of the cartridge holder. If the leaf spring is pushed radially inward, the adapter is thus pushed in the proximal direction relative to the adapter.

In an exemplary embodiment, an axial stop is arranged on the cartridge holder for abutting a needle hub of a needle assembly when it is screwed onto the adapter. As the needle hub proximally abuts the stop, it cannot follow the movement of the adapter when the spring is pushed radially inward and remains in its position relative to the cartridge holder. Despite the thread engagement between the adapter and the needle assembly, the internal thread can jump over the proximal ramped side of the thread segments allowing for pulling the adapter out of the needle hub. The needle assembly can thus be removed without rotation.

In an exemplary embodiment, the drug delivery device further comprises a cap adapted to be assembled over the cartridge holder thereby engaging the at least one leaf spring pushing it radially inwards against its bias thereby resiliently deforming the leaf spring such that it assumes a curved shape with a second curve radius greater than the first curve radius and moving the adapter in the proximal direction relative to the cartridge holder. The needle assembly is thus automatically removed from the cartridge and released from the adapter upon assembly of the cap.

In an exemplary embodiment, the leaf spring is adapted to resume its relaxed position on removal of the cap thus moving the adapter in the distal direction relative to the cartridge holder by the transversal side of the thread segments pushing against the internal thread of the needle hub. The needle assembly is thus automatically removed from the adapter upon removal of the cap.

An advantage of the device may be to provide a mechanism for reducing the risk of occluding an injection needle by an adapter for attaching a needle assembly to a cartridge holder,

Further scope of applicability of certain aspects, will become apparent from the detailed description given hereinafter. However, it should be understood that the detailed description and specific examples, while indicating exemplary embodiments of the invention, are given by way of illustration only, since various changes and modifications within the spirit and scope of the invention will become apparent to those skilled in the art from this detailed description.

Some embodiments will become more fully understood from the detailed description given herein below, wherein below and the accompanying drawings, which are given by way of illustration only, and do not limit the present invention.

### BRIEF DESCRIPTION OF THE FIGURES

FIG. 1 is a schematic perspective view of a cartridge holder and an adapter for attaching a needle assembly to the cartridge holder,

FIG. 2 is a schematic longitudinal section of the adapter attached to the cartridge holder in an extended position,

FIG. 3 is a schematic longitudinal section of the adapter with an attached needle assembly, wherein the adapter is attached to the cartridge holder in a snapped-in position,

FIG. 4 is a schematic longitudinal section of the adapter with the attached needle assembly and a cap assembled over the cartridge holder, the adapter and the needle assembly,

FIG. 5 is a schematic perspective view of another exemplary embodiment of a cartridge holder and an adapter for attaching a needle assembly to the cartridge holder,

FIG. 6 is a schematic longitudinal section of the adapter attached to the cartridge holder,

FIG. 7 is a schematic longitudinal section of the adapter with an attached needle assembly, wherein the adapter is attached to the cartridge holder, and

FIG. 8 is a schematic longitudinal section of the adapter with the attached needle assembly and a cap assembled over the cartridge holder, the adapter and the needle assembly.

Corresponding parts are marked with the same reference symbols in all figures.

### DETAILED DESCRIPTION

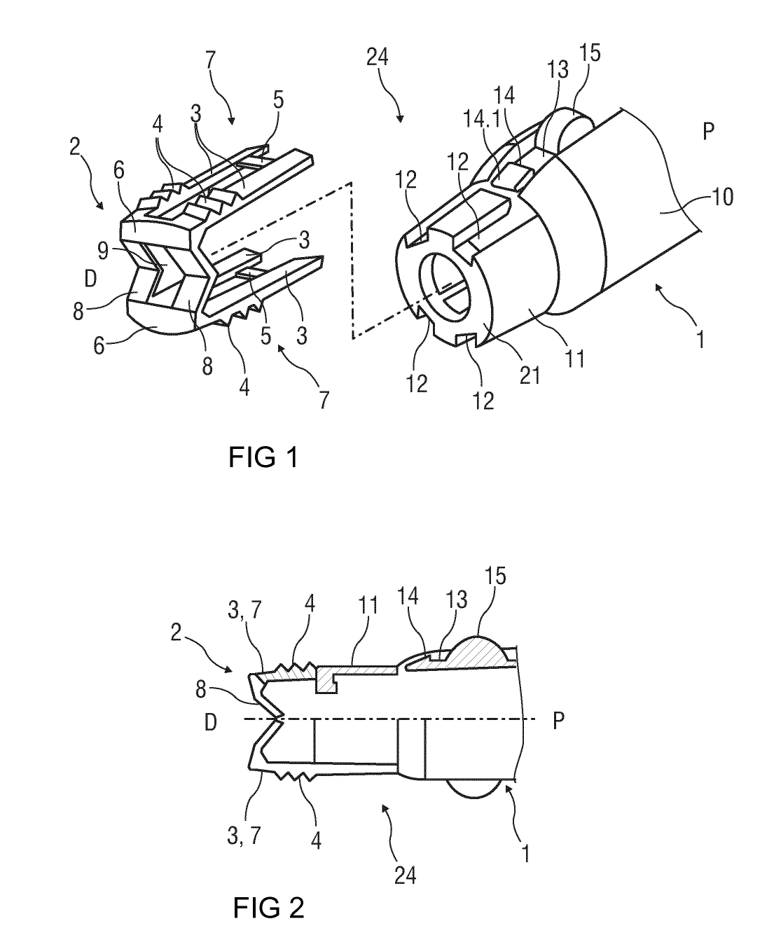

FIG. 1 is a schematic perspective view of a drug delivery device 24 comprising a cartridge holder 1 and an adapter 2 for attaching a needle assembly to the cartridge holder 1. The adapter 2 comprises four longitudinal arms 3 respectively having an external thread segment 4. Near a proximal end, two proximal connecting bars 5 are provided, each one rigidly interconnecting two of the arms 3. At a distal end, two distal connecting bars 6 are provided, each one rigidly interconnecting two of the arms 3 such that there are two pairs 7 of rigidly connected arms 3. The two pairs 7 are interconnected by two spring arms 8 at the distal end, the spring arms 8 being spaced from each other providing a central opening 9 in the adapter 2. The spring arms 8 allow for varying a distance between the pairs 7 of arms 3. The thread segments 4 are arranged to form an external thread adapted to engage an internal thread of a needle hub of a needle assembly (not illustrated). The spring arms 8 are adapted to pull the pairs 7 of arms 3 together.

The cartridge holder 1 comprises a body 10 adapted to receive a drug cartridge (not illustrated) arranged for containing a drug to be injected. At its distal end, the cartridge holder 1 comprises a substantially cylindrical interface 11 arranged to receive the adapter 2. The interface 11 comprises four longitudinal slots 12 adapted to guide the arms 3 of the adapter 2 such that the adapter 2 can slide relative to the cartridge holder 1 in a distal direction D and in a proximal direction P but is splined to the cartridge holder 1 preventing relative rotation between the adapter 2 and the cartridge holder 1. Furthermore, the cartridge holder 1 comprises two resilient arms 13 with a respective ramped snap hook 14 and a respective radially outwardly protruding cam 15. In a relaxed position of the resilient arm 13, the cam 15 protrudes from an outer surface of the body 10. The snap hook 14 is adapted to engage the proximal connecting bar 5 of the adapter 2 thus locking the adapter 2 in a snapped-in position.

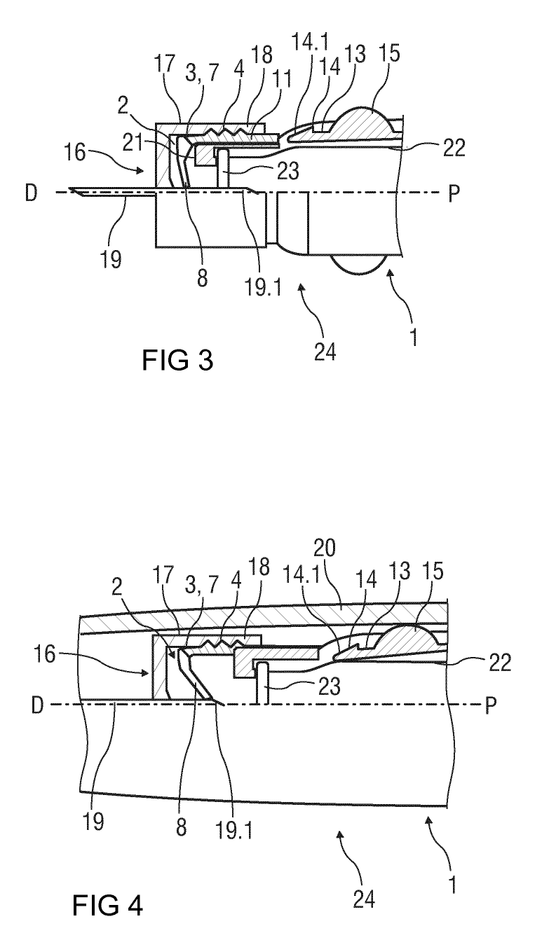

FIG. 2 is a schematic longitudinal section of the adapter 2 attached to the cartridge holder 1 in an extended position. The spring arms 8 pull the distal end of the pairs 7 of the arms 3 where the thread segments 4 are located together. As the proximal ends of the pairs 7 of arms 3 are located on the interface 11, the pairs 7 of arms 3 may be in an angled position tapering towards the distal direction D. In this state, the diameter of the screw thread defined by the thread segments 4 is smaller than a nominal diameter required for threadedly engaging an internal thread of a needle hub of a needle assembly. The proximal connecting bars 5 are located distally from the snap hooks 14 so that the adapter 2 is not locked in position relative to the cartridge holder 1. FIG. 3 is a schematic longitudinal section of the adapter 2 with an attached needle assembly 16, wherein the adapter 2 is attached to the cartridge holder 1 in a snapped-in position. The needle assembly 16 comprises a needle hub 17 having an internal thread 18. The needle assembly 16 further comprises a hypodermic double ended hollow needle 19 held in the needle hub 17. In order to attach the needle assembly 16 to the adapter 2 in the extended position as shown in FIG. 2, the needle hub 17 is pushed in the proximal direction P over the thread segments 4. Due to the reduced diameter of the thread segments 4 in the extended position of the adapter 2, the needle hub 17 does not have to be screwed on but can just slide onto the adapter 2. In this state, a proximal tip 19.1 of the needle 19 protruding through the central opening 9 is still axially spaced from a septum 23 of a drug cartridge 22 which may be held in the cartridge holder 1. Movement of the adapter 2 in the proximal direction P towards the cartridge holder 1 is opposed by the spring arms 8 biasing the distal ends of the pairs 7 of arms 3 together and abutting a distal face 21 of the cartridge holder 1 and by the proximal connecting bars 5 abutting distal ramps 14.1 of the snap hooks 14. As the needle hub 17 axially abuts the adapter 2, continued pushing of the needle assembly 16 in the proximal direction P results in movement of the adapter 2 in the proximal direction P relative to the cartridge holder 1. The distal ends of the pairs 7 of arms 3 are pulled apart against the bias of the spring arms 8 as the arms 3 ride up the slots 12. The external thread formed by the thread segments 4 thus takes up its nominal diameter and engages the internal thread 18 of the needle hub 17. At the same time, the proximal tip 19.1 of the needle 19 may pierce the septum 23 of the drug cartridge 22 establishing a fluid communication between the needle and a cavity provided in the cartridge. Furthermore, as during movement of the adapter 2 from the extended position shown in FIG. 2, the proximal connecting bars 5 ride up the ramp 14.1 of the snap hooks 14 until snapping into the snap hooks 14 and arriving in the snapped-in position shown in FIG. 3. The distal face 21 of the cartridge holder 1 located on the interface 11 may abut the spring arms 8, further straightening them against their bias thereby pushing the thread segments 4 further apart into their nominal position such that the needle assembly 16 is tightened on the adapter 2. The arms 3 are no longer angled but substantially in parallel to each other. A user may now perform an injection.

FIG. 4 is a schematic longitudinal section of the adapter 2 with the attached needle assembly 16 and a cap 20 assembled over the cartridge holder 1, the adapter 2 and the needle assembly 16. As, e.g. after an injection, the cap 20 is pushed over the cartridge holder 1, it engages the cams 15 deflecting them radially inwards along with the whole resilient arms 13 and the snap hooks 14. The snap hooks 14 thus disengage the proximal connecting bars 5. The tension stored in the spring arms 8 is thus released and pulls the adapter 2 with the needle assembly 16 back into the extended position pulling the proximal tip 19.1 out of the septum 23. The arms 3 take up their angled position again such that the diameter of the thread formed by the threaded segments 4 is reduced to such an extent that the needle hub 17 can be pulled off the adapter 2 when the cap 20 is subsequently removed. The cap 20 may comprise internal longitudinal ribs (not illustrated) for engaging the cams 15. The ribs may extend over part of the length of the cap 20 such that the cap 20 has to overlap a defined length of the cartridge holder 1 before releasing the adapter 2. Likewise, the ribs may extend over the whole length of the cap 20 or be just short segments at a proximal end of the cap 20 or the cap 20 may be arranged to engage the cams 15 with its internal surface.

The needle assembly 16 can thus be attached to the cartridge holder 1 without rotation such that the needle 19 does not have to be precisely centered in the septum 23 before. Removing the needle assembly 16 from the cartridge 22 may even be achieved without the user touching the needle.

FIG. 5 is a schematic perspective view of another exemplary embodiment of a drug delivery device 124 comprising a cartridge holder 101 and an adapter 102 for attaching a needle assembly to the cartridge holder 101. The adapter 102 comprises four longitudinal arms 103 (only two of them are illustrated) respectively having an external thread segment 104. Near a proximal end, two proximal connecting bars 105 (only one of them illustrated) having a transversal slot 105.1 are provided, each one rigidly interconnecting two of the arms 103 such that there are two pairs 107 of connected arms 103. The thread segments 104 are arranged to form an external thread adapted to engage an internal thread of a needle hub of a needle assembly (not illustrated). The thread segments 104 comprise a proximal ramped side 104.1 at a relatively acute angle relative to a longitudinal axis L and a distal transversal side 104.2 at right angles or at a more obtuse angle relative to the longitudinal axis L such that the thread segments 104 exhibit a saw tooth profile. The arms 103 may exhibit a dove tail cross section being wider on a radially inwards pointing side than on a radially outward pointing side.

The cartridge holder 101 comprises a body 110 adapted to receive a drug cartridge (not illustrated) arranged for containing a drug to be injected. At its distal end, the cartridge holder 101 comprises a substantially cylindrical interface 111 arranged to receive the adapter 102. The interface 111 comprises four longitudinal slots 112 adapted to guide the arms 103 of the adapter 102 such that the adapter 102 can slide relative to the cartridge holder 101 in a distal direction D and in a proximal direction P but is splined to the cartridge holder 101 preventing relative rotation between the adapter 102 and the cartridge holder 101. The slots 112 may comprise a dove tail cross section corresponding to the cross section of the arms 103. Further longitudinal slots 113 in the cartridge holder 101 may be arranged for accommodating and guiding a proximal end of one pair 107 of arms 103, respectively.

Two leaf springs 108 are arranged (only one of them illustrated), each one grounded distally in a spring seat 109 of the cartridge holder 101 and proximally in the transversal slot 105.1 of one of the connecting bars 105. In a relaxed state, as illustrated, the leaf spring 108 has a curved shape with a relatively small first curve radius biasing the arms 103 in the distal direction D.

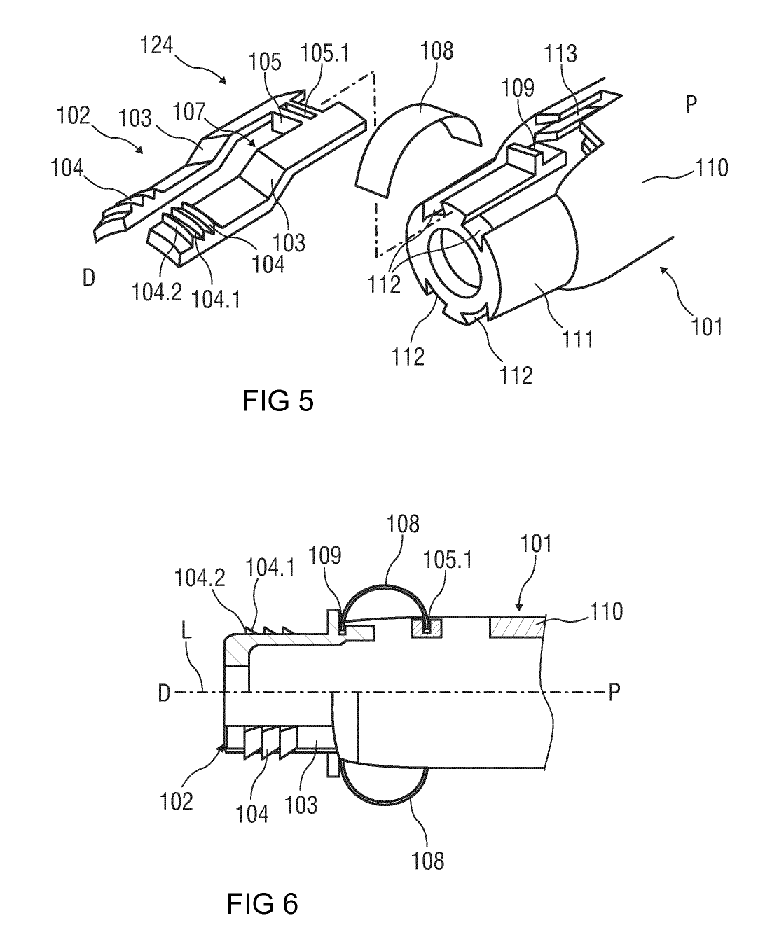

FIG. 6 is a schematic longitudinal section of the adapter 102 attached to the cartridge holder 101. The leaf springs 108 are in their relaxed state with the first curve radius biasing the arms 103 in the distal direction D. In the relaxed state, the leaf springs 108 protrude radially outwardly beyond a surface of the cartridge holder 101.

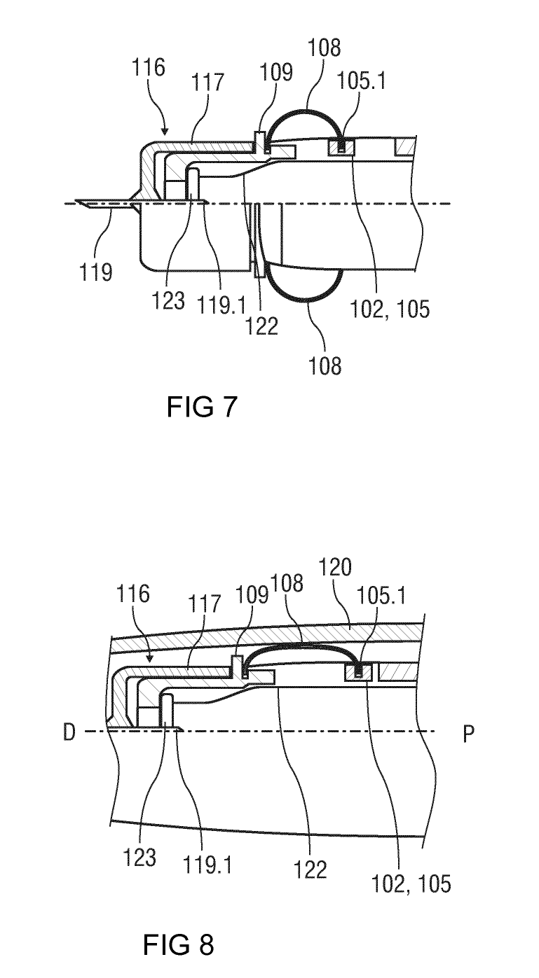

FIG. 7 is a schematic longitudinal section of the adapter 102 with an attached needle assembly 116, wherein the adapter 102 is attached to the cartridge holder 101. The needle assembly 116 comprises a needle hub 117 having an internal thread (not illustrated). The needle assembly 116 further comprises a hypodermic double ended hollow needle 119 held in the needle hub 117. In order to attach the needle assembly 116 to the adapter 102, as shown in FIG. 6, the needle hub 117 is screwed onto the thread segments 104 until the needle hub 117 abuts a stop on the cartridge holder 101 which may be part of the spring seat 109. At the same time, a proximal tip 119.1 of the needle 119 may pierce a septum 123 of a drug cartridge 122 establishing a fluid communication between the needle 119 and a cavity provided in the cartridge 122. A user may now perform an injection.

FIG. 8 is a schematic longitudinal section of the adapter 102 with the attached needle assembly 116 and a cap 120 assembled over the cartridge holder 101, the adapter 102 and the needle assembly 116. As, e. g. after an injection, the cap 120 is pushed over the cartridge holder 101, it engages the leaf springs 108 pushing them radially inwards against their bias. The ends of the leaf spring 108 grounded in the cartridge holder 101 and in the adapter 102 are thus moved apart such that the leaf spring 108 has a curved shape with a second curve radius greater than the first curve radius and the adapter 102 is moved in the proximal direction P relative to the cartridge holder 101. As the needle hub 117 proximally abuts the stop on the spring seat 109, it cannot follow the movement of the adapter 102 and remains in its relative position relative to the cartridge holder 101. Despite the thread engagement between the adapter 102 and the needle assembly 116, the internal thread can jump over the proximal ramped side 104.1 of the thread segments 104 allowing for pulling the adapter 102 out of the needle hub 117.

The cap 120 may comprise internal longitudinal ribs (not illustrated) for engaging the leaf springs 108. The ribs may extend over part of the length of the cap 120 such that the cap 120 has to overlap a defined length of the cartridge holder 101 before deflecting the leaf spring 108. Likewise, the ribs may extend over the whole length of the cap 120 or be just short segments at a proximal end of the cap 120 or the cap 120 may be arranged to engage the leaf springs 108 with its internal surface.

If the cap 120 is subsequently removed, the leaf springs 108 will resume their relaxed position as in FIG. 6 moving the adapter 102 in the distal direction D relative to the cartridge holder 101. Due to the transversal side 104.2 of the thread segments 104 and the needle hub 117 now being rather loosely arranged on the interface 111, the thread segments 104 will not re-engage the internal thread of the needle hub 117 but push it in the distal direction D off the interface 111.

Removing the needle assembly 116 from the cartridge 122 may thus be achieved without the user touching the needle.

The illustrated embodiments described above exhibit adapters 2, 102 with four arms 3, 103. The skilled person will understand that the adapter 2, 102 may likewise comprise a different number of arms 3, 103, e. g. one, two, three or more than four arms 3, 103. Furthermore, the snap hook 14 and the leaf spring 108 may engage the adapter 2, 102 on only one respective arm 3, 103 instead of at a proximal connecting bar 5, 105.

The term “drug” or “medicament”, as used herein, means a pharmaceutical formulation containing at least one pharmaceutically active compound,

wherein in one embodiment the pharmaceutically active compound has a molecular weight up to 1500 Da and/or is a peptide, a protein, a polysaccharide, a vaccine, a DNA, a RNA, an enzyme, an antibody or a fragment thereof, a hormone or an oligonucleotide, or a mixture of the above-mentioned pharmaceutically active compound,
wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,
wherein in a further embodiment the pharmaceutically active compound comprises at least one peptide for the treatment and/or prophylaxis of diabetes mellitus or complications associated with diabetes mellitus such as diabetic retinopathy,
wherein in a further embodiment the pharmaceutically active compound comprises at least one human insulin or a human insulin analogue or derivative, glucagon-like peptide (GLP-1) or an analogue or derivative thereof, or exendin-3 or exendin-4 or an analogue or derivative of exendin-3 or exendin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-Nmyristoyl LysB28ProB29 human insulin; B28-N-palmitoyl-LysB28ProB29 human insulin; B30-Nmyristoyl-ThrB29LysB30 human insulin; B30-N-palmitoyl-ThrB29LysB30 human insulin; B29-N(N-palmitoyl-Y-glutamyl)-des(B30) human insulin; B29-N—(N-lithocholyl-Y-glutamyl)-des(B30) human insulin; B29-N-(ω-carboxyheptadecanoyl)-des(B30) human insulin and B29-N-(ωcarboxyheptadecanoyl) human insulin.

Exendin-4 for example means Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-LeuLys-Asn-Gly-Gly-Pro-Ser-Ser-Gly-Ala-Pro-Pro-Pro-Ser-NH2.

Exendin-4 derivatives are for example selected from the following list of compounds:

H-(Lys)4-des Pro36, des Pro37 Exendin-4(1-39)-NH2, H-(Lys)5-des Pro36, des Pro37 Exendin-4(1-39)-NH2, des Pro36 Exendin-4(1-39), des Pro36 [Asp28] Exendin-4(1-39), des Pro36 [IsoAsp28] Exendin-4(1-39), des Pro36 [Met(O)14, Asp28] Exendin-4(1-39), des Pro36 [Met(O)14, IsoAsp28] Exendin-4(1-39), des Pro36 [Trp(O2)25, Asp28] Exendin-4(1-39), des Pro36 [Trp(O2)25, IsoAsp28] Exendin-4(1-39), des Pro36 [Met(O)14 Trp(O2)25, Asp28] Exendin-4(1-39), des Pro36 [Met(O)14 Trp(O2)25, IsoAsp28] Exendin-4(1-39); or des Pro36 [Asp28] Exendin-4(1-39), des Pro36 [IsoAsp28] Exendin-4(1-39), des Pro36 [Met(O)14, Asp28] Exendin-4(1-39), des Pro36 [Met(O)14, IsoAsp28] Exendin-4(1-39), des Pro36 [Trp(O2)25, Asp28] Exendin-4(1-39), des Pro36 [Trp(O2)25, IsoAsp28] Exendin-4(1-39), des Pro36 [Met(O)14 Trp(O2)25, Asp28] Exendin-4(1-39), des Pro36 [Met(O)14 Trp(O2)25, IsoAsp28] Exendin-4(1-39),
wherein the group -Lys6-NH2 may be bound to the C-terminus of the Exendin-4 derivative;
or an Exendin-4 derivative of the sequence
des Pro36 Exendin-4(1-39)-Lys6-NH2 (AVE0010), H-(Lys)6-des Pro36 [Asp28] Exendin-4(1-39)-Lys6-NH2, des Asp28 Pro36, Pro37, Pro38Exendin-4(1-39)-NH2, H-(Lys)6-des Pro36, Pro38 [Asp28] Exendin-4(1-39)-NH2, H-Asn-(Glu)5des Pro36, Pro37, Pro38 [Asp28] Exendin-4(1-39)-NH2, des Pro36, Pro37, Pro38 [Asp28] Exendin-4(1-39)-(Lys)6-NH2, H-(Lys)6-des Pro36, Pro37, Pro38 [Asp28] Exendin-4(1-39)-(Lys)6-NH2, H-Asn-(Glu)5-des Pro36, Pro37, Pro38 [Asp28] Exendin-4(1-39)-(Lys)6-NH2, H-(Lys)6-des Pro36 [Trp(O2)25, Asp28] Exendin-4(1-39)-Lys6-NH2, H-des Asp28 Pro36, Pro37, Pro38 [Trp(O2)25] Exendin-4(1-39)-NH2, H-(Lys)6-des Pro36, Pro37, Pro38 [Trp(O2)25, Asp28] Exendin-4(1-39)-NH2, H-Asn-(Glu)5-des Pro36, Pro37, Pro38 [Trp(O2)25, Asp28] Exendin-4(1-39)-NH2, des Pro36, Pro37, Pro38 [Trp(O2)25, Asp28] Exendin-4(1-39)-(Lys)6-NH2, H-(Lys)6-des Pro36, Pro37, Pro38 [Trp(O2)25, Asp28] Exendin-4(1-39)-(Lys)6-NH2, H-Asn-(Glu)5-des Pro36, Pro37, Pro38 [Trp(O2)25, Asp28] Exendin-4(1-39)-(Lys)6-NH2, H-(Lys)6-des Pro36 [Met(O)14, Asp28] Exendin-4(1-39)-Lys6-NH2, des Met(O)14 Asp28 Pro36, Pro37, Pro38 Exendin-4(1-39)-NH2, H-(Lys)6-desPro36, Pro37, Pro38 [Met(O)14, Asp28] Exendin-4(1-39)-NH2, H-Asn-(Glu)5-des Pro36, Pro37, Pro38 [Met(O)14, Asp28] Exendin-4(1-39)-NH2, des Pro36, Pro37, Pro38 [Met(O)14, Asp28] Exendin-4(1-39)-(Lys)6-NH2, H-(Lys)6-des Pro36, Pro37, Pro38 [Met(O)14, Asp28] Exendin-4(1-39)-(Lys)6-NH2, H-Asn-(Glu)5 des Pro36, Pro37, Pro38 [Met(O)14, Asp28] Exendin-4(1-39)-(Lys)6-NH2, H-Lys6-des Pro36 [Met(O)14, Trp(O2)25, Asp28] Exendin-4(1-39)-Lys6-NH2, H-des Asp28 Pro36, Pro37, Pro38 [Met(O)14, Trp(O2)25] Exendin-4(1-39)-NH2, H-(Lys)6-des Pro36, Pro37, Pro38 [Met(O)14, Asp28] Exendin-4(1-39)-NH2, H-Asn-(Glu)5-des Pro36, Pro37, Pro38 [Met(O)14, Trp(O2)25, Asp28] Exendin-4(1-39)-NH2, des Pro36, Pro37, Pro38 [Met(O)14, Trp(O2)25, Asp28] Exendin-4(1-39)-(Lys)6-NH2, H-(Lys)6-des Pro36, Pro37, Pro38 [Met(O)14, Trp(O2)25, Asp28] Exendin-4(S1-39)-(Lys)6-NH2, H-Asn-(Glu)5-des Pro36, Pro37, Pro38 [Met(O)14, Trp(O2)25, Asp28] Exendin-4(1-39)-(Lys)6-NH2;
or a pharmaceutically acceptable salt or solvate of any one of the afore-mentioned Exendin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra-low molecular weight heparin or a derivative thereof, or a sulphated, e.g. a poly-sulphated form of the above-mentioned polysaccharides, and/or apharmaceutically acceptable salt thereof. An example of a pharmaceutically acceptable salt of a poly-sulphated low molecular weight heparin is enoxaparin sodium.

Antibodies are globular plasma proteins (˜150 kDa) that are also known as immunoglobulins which share a basic structure. As they have sugar chains added to amino acid residues, they are glycoproteins. The basic functional unit of each antibody is an immunoglobulin (Ig) monomer (containing only one Ig unit); secreted antibodies can also be dimeric with two Ig units as with IgA, tetrameric with four Ig units like teleost fish IgM, or pentameric with five Ig units, like mammalian IgM.

The Ig monomer is a “Y”-shaped molecule that consists of four polypeptide chains; two identical heavy chains and two identical light chains connected by disulfide bonds between cysteine residues. Each heavy chain is about 440 amino acids long; each light chain is about 220 amino acids long. Heavy and light chains each contain intrachain disulfide bonds which stabilize their folding. Each chain is composed of structural domains called Ig domains. These domains contain about 70-110 amino acids and are classified into different categories (for example, variable or V, and constant or C) according to their size and function. They have a characteristic immunoglobulin fold in which two β sheets create a “sandwich” shape, held together by interactions between conserved cysteines and other charged amino acids.

There are five types of mammalian Ig heavy chain denoted by α, δ, ε, γ, and μ. The type of heavy chain present defines the isotype of antibody; these chains are found in IgA, IgD, IgE, IgG, and IgM antibodies, respectively.

Distinct heavy chains differ in size and composition; α and γ contain approximately 450 amino acids and δ approximately 500 amino acids, while μ and ε have approximately 550 amino acids. Each heavy chain has two regions, the constant region (CH) and the variable region (VH). In one species, the constant region is essentially identical in all antibodies of the same isotype, but differs in antibodies of different isotypes. Heavy chains γ, α and δ have a constant region composed of three tandem Ig domains, and a hinge region for added flexibility; heavy chains μ and c have a constant region composed of four immunoglobulin domains. The variable region of the heavy chain differs in antibodies produced by different B cells, but is the same for all antibodies produced by a single B cell or B cell clone. The variable region of each heavy chain is approximately 110 amino acids long and is composed of a single Ig domain.

In mammals, there are two types of immunoglobulin light chain denoted by λ and κ. A light chain has two successive domains: one constant domain (CL) and one variable domain (VL). The approximate length of a light chain is 211 to 217 amino acids. Each antibody contains two light chains that are always identical; only one type of light chain, κ or λ, is present per antibody in mammals.

Although the general structure of all antibodies is very similar, the unique property of a given antibody is determined by the variable (V) regions, as detailed above. More specifically, variable loops, three on the light (VL) and three on the heavy (VH) chain, are responsible for binding to the antigen, i.e. for its antigen specificity. These loops are referred to as the Complementarity Determining Regions (CDRs). Because CDRs from both VH and VL domains contribute to the antigen-binding site, it is the combination of the heavy and the light chains, and not either alone, that determines the final antigen specificity.

An “antibody fragment” contains at least one antigen binding fragment as defined above, and exhibits essentially the same function and specificity as the complete antibody of which the fragment is derived from. Limited proteolytic digestion with papain cleaves the Ig prototype into three fragments. Two identical amino terminal fragments, each containing one entire L chain and about half an H chain, are the antigen binding fragments (Fab). The third fragment, similar in size but containing the carboxyl terminal half of both heavy chains with their interchain disulfide bond, is the crystalizable fragment (Fc). The Fc contains carbohydrates, complement binding, and FcR-binding sites. Limited pepsin digestion yields a single F(ab′)2 fragment containing both Fab pieces and the hinge region, including the H—H interchain disulfide bond. F(ab′)2 is divalent for antigen binding. The disulfide bond of F(ab′)2 may be cleaved in order to obtain Fab′. Moreover, the variable regions of the heavy and light chains can be fused together to form a single chain variable fragment (scFv).

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates. Those of skill in the art will understand that modifications (additions and/or removals) of various components of the apparatuses, methods and/or systems and embodiments described herein may be made without departing from the full scope and spirit of the present invention, which encompass such modifications and any and all equivalents thereof.

### LIST OF REFERENCES

1 cartridge holder
2 adapter
3 arm
4 thread segment
5 proximal connecting bar
6 distal connecting bar
7 pair
8 spring arm
9 central opening
10 body
11 interface
12 longitudinal slot
13 resilient arm
14 snap hook
14.1 distal ramp
15 cam
16 needle assembly
17 needle hub
18 internal thread
19 needle
19.1 proximal tip
20 cap
21 distal face
22 cartridge
23 septum
24 drug delivery device
101 cartridge holder
102 adapter
103 arm
104 thread segment
104.1 ramped side
104.2 transversal side
105 proximal connecting bar
105.1 transversal slot
107 pair
108 leaf spring
109 spring seat
110 body
111 interface
112 longitudinal slot
113 further longitudinal slot
116 needle assembly
117 needle hub
119 needle
119.1 proximal tip
120 cap
122 cartridge
123 septum
124 drug delivery device
D distal direction
L longitudinal axis
P proximal direction

1-15. (canceled)

16. An adapter for attaching a needle assembly to a cartridge holder, the adapter comprising at least one longitudinal arm adapted to engage one or more respective longitudinal slots in the cartridge holder, such that the adapter is slidable relative to the cartridge holder, the at least one arm having an external thread segment adapted to engage an internal thread of a needle hub of the needle assembly, wherein the at least one arm is adapted to allow for rotationless disengaging the thread segment from the internal thread.

17. The adapter according to claim 16, wherein the adapter comprises at least two arms, at least two of the arms interconnected to form one or more pairs of connected arms.

18. The adapter according to claim 17, wherein at least two arms or two pairs are interconnected by one or more spring arms at a distal end, the spring arms being configured to pull the distal end of the arms or pairs together thus reducing a diameter of a screw thread defined by the thread segments to be smaller than a nominal diameter corresponding to the internal thread of the needle hub of the needle assembly.

19. The adapter according to claim 16, wherein the thread segment exhibits a saw tooth profile comprising a proximal ramped side and a distal transversal side.

20. A drug delivery device, comprising
a cartridge holder adapted to receive a drug cartridge comprising an interface with one or more longitudinal slots; and
an adapter comprising at least one longitudinal arm adapted to engage the one or more longitudinal slots of the cartridge holder, such that the adapter is slidable relative to the cartridge holder in a distal direction and in a proximal direction and splined to the cartridge holder, the at least one longitudinal arm having an external thread segment adapted to engage an internal thread of a needle hub of a needle assembly, wherein the at least one arm is adapted to allow for rotationless disengaging the thread segment from the internal thread,
wherein the interface of the cartridge holder is arranged to receive the adapter such that the one or more longitudinal slots of the cartridge holder guide the at least one arm of the adapter, and wherein a number of longitudinal slots of the cartridge holder is equal to a number of longitudinal arms of the adapter.

21. The drug delivery device according to claim 20, wherein the at least one arm exhibits a dove tail cross section being wider on a radially inwards pointing side than on a radially outward pointing side, wherein the one or more slots comprises a dove tail cross section corresponding to the cross section of the at least one arm.

22. The drug delivery device according to claim 20, wherein the cartridge holder comprises at least one resilient arm with a ramped snap hook and a radially outwardly protruding cam, wherein in a relaxed position of the resilient arm the cam protrudes from an outer surface of a body of the cartridge holder, wherein the snap hook is adapted to engage the adapter for locking it in a snapped-in position.

23. The drug delivery device, according to claim 22, wherein at least two arms or two pairs are interconnected by spring arms at a distal end, the spring arms pull the distal end of the arms or pairs, thereby creating a bias on the arms or pairs due to the spring arms, and wherein when the adapter is in an extended position distally from the snapped-in position, the spring arms pull the distal end of the arms or pairs together, thus reducing a diameter of a screw thread defined by the thread segments to become smaller than a nominal diameter corresponding to the internal thread of the needle hub of the needle assembly,

24. The drug delivery device according to claim 23, wherein a distal face of the cartridge holder is arranged to abut the spring arm opposing movement of the adapter from the extended position in a proximal direction relative to the cartridge holder, wherein the distal ends of the arms or pairs of the cartridge holder are pulled apart against the bias created by the spring arms as the arms of the cartridge holder ride up the slots when the adapter is moved towards the snapped-in position causing the external thread formed by the thread segments expand to the nominal diameter corresponding to the internal thread of the needle hub.

25. The drug delivery device according to claim 24, wherein the distal face is adapted to abut the at least one spring arm straightening it against the bias created by the spring arms thereby pushing the thread segments further apart causing the external thread formed by the thread segments to expand to the nominal diameter.

26. The drug delivery device according to claim 24, further comprising a cap adapted to be assembled over the cartridge holder thereby engaging the cam deflecting the resilient arm radially inwards thus disengaging the snap hook from the adapter allowing it to return into the extended position due to tension stored in the spring arms thereby reducing the diameter of the screw thread defined by the thread segments to become smaller than the nominal diameter.

27. The drug delivery device according to claim 20, wherein an axial stop is arranged on the cartridge holder for abutting the needle hub of the needle assembly when it is screwed onto the adapter.

28. The drug delivery device according to claim 20, wherein at least one leaf spring is grounded distally in the cartridge holder and proximally in the adapter, wherein in a relaxed state the leaf spring has a curved shape with a first curve radius biasing the adapter in the distal direction, wherein in the relaxed state the leaf spring protrudes radially outwardly beyond a surface of the cartridge holder.

29. The drug delivery device according claim 28, further comprising a cap adapted to be assembled over the cartridge holder thereby engaging the at least one leaf spring pushing it radially inwards against its bias thereby resiliently deforming the at least one leaf spring such that it assumes a curved shape with a second curve radius greater than the first curve radius and moving the adapter in the proximal direction relative to the cartridge holder.

30. The drug delivery device according to claim 29, wherein the leaf spring is adapted to resume the relaxed position on removal of the cap thus moving the adapter in the distal direction relative to the cartridge holder by a transversal side of the thread segments pushing against the internal thread of the needle hub.

31. A method comprising:
sliding an adapter and a needle assembly relative to each other so that the needle assembly surrounds the adapter; and
pushing the needle hub so that arms of the adapter flex radially outward and secure the needle hub to the adapter.

32. The method of claim 31, wherein the radially flexing arms of the adapter engage internal threads of the needle hub with external threads of the adapter.

33. The method of claim 31, further comprising removing the needle assembly by sliding a cap over a cartridge holder coupled to the adapter such that the cartridge holder disengages from the adapter and the adapter disengages from the needle hub.

34. The method of claim 33, wherein removing the needle assembly further comprises pulling the needle assembly axially from the adapter.
